(19) United States
(12) Patent Application Publication
(10) Pub. No.: US 2014/0341882 A1
(43) Pub. Date: Nov. 20, 2014

(54) PHARMACEUTICAL PRODUCT FOR PREVENTING OR TREATING ALZHEIMER'S DISEASE

(71) Applicant: Takahiro Ochiya, Chuo-ku (JP)

(72) Inventor: Takahiro Ochiya, Chuo-ku (JP)

(21) Appl. No.: 14/344,420

(22) PCT Filed: Sep. 7, 2012
(86) PCT No.: PCT/JP2012/072909

§ 371 Date: Mar. 12, 2014

(30) Foreign Application Priority Data
Sep. 13, 2011 (JP) 2011-199772

(51) Int. Cl. A61K 38/48

(52) U.S. Cl. CPC A61K 38/4886; C12Y 304/24011
USPC 424/94.67; 435/219

(57) ABSTRACT

The present invention provides a therapeutic agent for Alzheimer's disease. The therapeutic agent contains membrane vesicles (exosomes) of adipose tissue-derived mesenchymal stem cells, and the membrane vesicles (exosomes) contain neprilysin. Through the research by the inventors of the present invention, it was revealed that exosomes secreted by human adipose tissue-derived mesenchymal stem cells contain neprilysin, which degrades amyloid-β as a pathogenic protein of Alzheimer's disease. When exosomes secreted by human adipose tissue-derived mesenchymal stem cells were administered to the brains of Alzheimer's disease model mice, the generation of amyloid-β was inhibited.

### TECHNICAL FIELD

The present invention relates to a pharmaceutical product for preventing or treating Alzheimer's disease.

### BACKGROUND ART

Alzheimer's disease is the most common senile dementia across the world, and is characterized by progressive cognitive impairments (such as memory problems, disorientation, learning disability, and space perception impairment). In a person with Alzheimer's disease, amyloid plaques are seen in the cerebral cortex, and amyloid-β is the main component of the amyloid plaques. Various therapeutic agents for Alzheimer's disease have been proposed (for example, Patent Document 1). Unfortunately, however, no effective therapeutic agent is currently present.

### CITATION LIST

### Patent Document(s)

Patent Document 1: JP 2011-126847 A

### SUMMARY OF THE INVENTION

### Problem to be Solved by the Invention

With the foregoing in mind, it is an object of the present invention to provide a pharmaceutical product effective for preventing or treating Alzheimer's disease, and a method for preventing or treating Alzheimer's disease.

### Means for Solving Problem

The present invention provides a pharmaceutical product for preventing or treating Alzheimer's disease, containing: a membrane vesicle of an adipose tissue-derived mesenchymal stem cell.

The present invention also provides a method for preventing or treating Alzheimer's disease, including: administering a membrane vesicle of adipose tissue-derived mesenchymal stem cell to an Alzheimer's disease patient.

The membrane vesicle contains neprilysin.

### Effects of the Invention

The inventors of the present invention found out that neprilysin, which degrades amyloid-βis present in membrane vesicles of adipose tissue-derived mesenchymal stem cells, and also found out, by administering the membrane vesicles, amyloid-β is degraded so that Alzheimer's disease can be prevented or treated. Based on these findings, the inventors of the present invention achieved the present invention. According to the present invention, it is possible to provide a pharmaceutical product effective for preventing or treating Alzheimer's disease and a method for preventing or treating Alzheimer's disease.

### BRIEF DESCRIPTION OF DRAWINGS

FIG. 1 shows electrophoretograms indicating the presence of neprilysin in membrane vesicles (exosomes) of adipose tissue-derived mesenchymal stem cells.

FIG. 2 shows graphs showing the activity of neprilysin in membrane vesicles (exosomes) of adipose tissue-derived mesenchymal stem cells.

FIG. 3 shows photographs of tissues, demonstrating that amyloid-β disappeared in mice to which membrane vesicles (exosomes) of adipose tissue-derived mesenchymal stem cells had been administered.

### MODE FOR CARRYING OUT THE INVENTION

In the present invention, the adipose tissue-derived mesenchymal stem cells are not particularly limited, and may be derived from an Alzheimer's disease patient or a humans who is not an Alzheimer's disease patient, for example. For example, the pharmaceutical product according to the present invention may be prepared using adipose tissue-derived mesenchymal stem cells collected from an Alzheimer's disease patient to be treated. Alternatively, the pharmaceutical product according to the present invention for an Alzheimer's disease patient to be treated may be prepared using adipose tissue-derived mesenchymal stem cells collected from any other person.

In the present invention, the membrane vesicle is a membranous substance secreted by an adipose tissue-derived mesenchymal stem cell, and the size thereof is, for example, 5 to 200 nm, preferably 10 to 100 nm in diameter. The membrane vesicle is an exosome, for example. The exosome refers to, among various vesicles present in an intracellular membrane, a nanometer-order vesicle secreted to the outside of the cell, for example.

In the present invention, membrane vesicles containing neprilysin can be produced by culturing adipose tissue-derived mesenchymal stem cells, for example. A method for collecting the membrane vesicles from the cultured adipose tissue-derived mesenchymal stem cells is not particularly limited. For example, membrane vesicles released to the outside of the cells may be collected by collecting the culture supernatant, and membrane vesicles present inside the cells may be collected after disrupting the cells.

Collection of the membrane vesicles can be achieved by, for example, filtration, centrifugation, or the like. The membrane vesicles can be fractionated on the basis of their diameters, for example. As a specific example, the exosomes can be fractionated on the basis of the diameter in the range from 30 to 100 nm, for example. Specific examples of the collection method include a method in which a culture solution is centrifuged (for example, 500×g, 10 minutes) to collect the supernatant, the supernatant is filtered (for example, using a 0. 22-μm filter) and then ultracentrifuged (for example, 100,000×g to 120,000×g, 70 minutes), and the precipitated fraction is collected as membrane vesicles. Also, the membrane vesicles may be collected based on a density gradient using carriers, such as sucrose, with different specific gravities and then performing density gradient centrifugation. The collection of the membrane vesicles also can be carried out with reference to literatures such as Nat. Cell. Biol., 2007 June, 9(6), pp. 654-659; Epub 2007 May 7, PMID: 17486113; and the like.

In the present invention, a method for administering the membrane vesicles is not particularly limited, and examples thereof include administration by injection, transdermal administration, and oral administration. The administration by injection may be, for example, subcutaneous injection, intravenous injection, or the like. The dose is determined as appropriate depending on the body size, age, sex, progress of the disease, etc. of the patient.

### EXAMPLES

Next, examples of the present invention will be described. It is to be noted, however, that the present invention is by no means restricted or limited by the following examples.

### CONFIRMATION OF THE PRESENCE OF NEPRILYSIN

The presence of neprilysin in membrane vesicles of adipose tissue-derived mesenchymal stem cells was confirmed by the following method.

Human adipose tissue-derived mesenchymal stem cells (1×10⁶/20 ml) were cultured in a serum-free medium for 72 hours, after which the culture supernatant was collected. This culture supernatant was centrifuged at 2,000×g for 10 minutes, and cell debris and the like were removed. Thereafter, the culture supernatant further was ultracentrifuged at 100,000×g (4° C.) for 70 minutes. The supernatant was then discarded, and physiological saline was added to the residue to cause precipitation. The thus-obtained precipitated fraction was used as an exosome fraction. This exosome fraction was dissolved so that the total amount of the resultant mixture was 200 ml, and the exosome fraction was purified by ultracentrifugation. The protein amount was quantified by the Bradford protein assay, and a 10 mg protein equivalent per well was used in the Western blotting. After performing PAGE using Mini-PROTEAN TGX gels of 4% to 15% (Bio-Rad), the separated proteins were transferred on PVDF membranes and subjected to blocking at 4° C. overnight (Nacalai blocking solution). Next, a primary antibody reaction was carried out for 1 hour at room temperature (mouse monoclonal anti-CD10, ab951, 1:1000). Subsequently, a secondary antibody reaction was carried out for 1 hour at room temperature (sheep anti-mouse Ig HRP conjugated, 1:5000). At the end, a color-developing reaction was carried out using an Immunostar (Wako) (for 5 minutes at room temperature), and the blotting images were photographed.

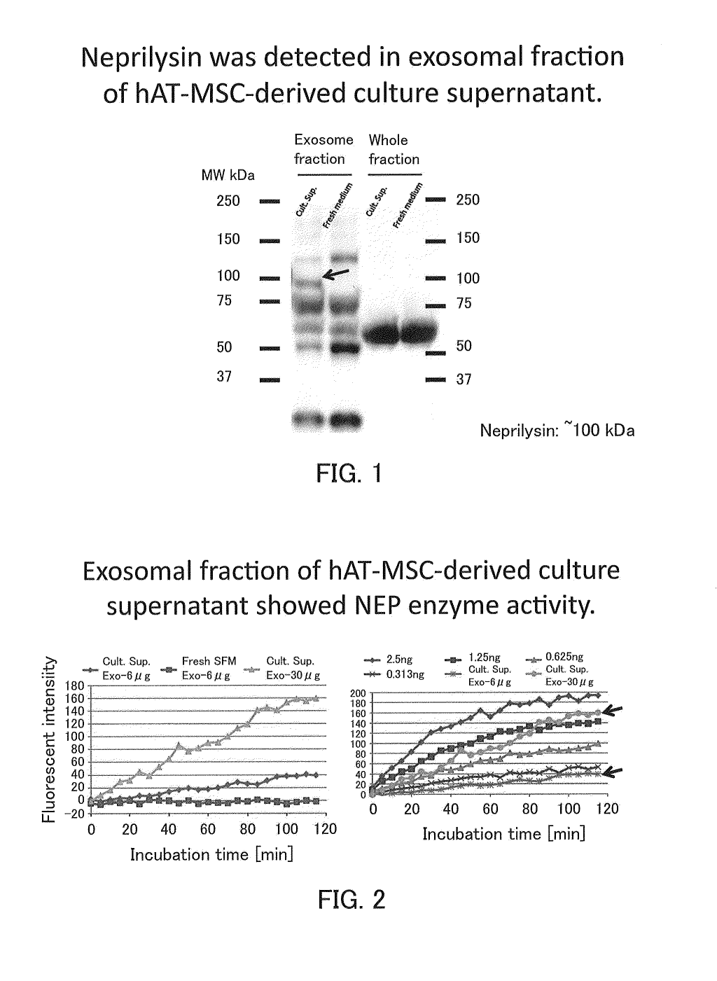

The photograph in FIG. 1 shows the result of the above-described blotting. In the photograph in FIG. 1, the band indicated with the arrow corresponds to neprilysin. This result confirmed that neprilysin is present in membrane vesicles (exosomes) of human adipose tissue-derived mesenchymal stem cells.

### CONFIRMATION OF ACTIVITY OF NEPRILYSIN

Next, the activity of the neprilysin in the membrane vesicles (exosomes) obtained in the above-described manner was confirmed by the following method.

The above-described exosome fraction was dissolved in an assay buffer (100 mM Tris, 50 mM NaCl, 10 mg/mL ZnCl₂, pH=7.5) to prepare a sample. Also, as standard samples, solutions containing recombinant human Neprilysin (R&D) at the respective concentrations in the range from 0.31 ng to 10 ng were used. Each sample was supplied to a 96-well black plate on ice (6 ug/well and 30 μg/well). Subsequently, an assay buffer containing a 20 mM substrate and 10mg/mL thiorphan (±) was added so that the respective wells contained equal amounts (50 mL) of the assay buffer, and then the measurement was started. During the measurement, the fluorescence intensity was measured every 5 minutes while incubating the plate at 37° C., and the kinetics of the enzyme reaction was observed.

The neprilysin activity was measured using a fluorogenic substrate Mca-RPPGFSAFK (Dnp). This is not only a substrate for neprilysin but also a substrate for several kinds of other enzymes. Thus, in the measurement of the neprilysin activity, a system containing thiorphan, which is an inhibitor specific to neprilysin, also was provided, and the activity of the enzymes other than neprilysin also was measured. The activity of the neprilysin was evaluated according to the following equation.

Neprilysin activity=fluorescence intensity (in the absence of inhibitor)−fluorescence intensity (in the presence of inhibitor)

The graphs in FIG. 2 show the results of the neprilysin activity measurement. The neprilysin activity measurement was carried out through observation for 120 minutes after the addition of the fluorogenic substrate. When the exosomes derived from the human adipose tissue-derived mesenchymal stem cells (hAT-MSC) were added to the solution containing the fluorogenic substrate and the resultant mixture was incubated at 37° C., an increase in fluorescence intensity over time was observed, from which it was confirmed that the exosomes have enzyme activity. In contrast, in the system containing the inhibitor specific to neprilysin (NEP), the increase in fluorescence intensity decreased considerably. This revealed that the exosomes derived from the human adipose tissue-derived mesenchymal stem cells (hAT-MSC) have neprilysin (NEP) activity. Also, from the comparison with the dilution series of the recombinant human neprilysin (rhNEP), it was found that the activity of the neprilysin (NEP) derived from these exosomes was approximately equivalent to the activity of 1.25 ng of rhNEP.

### INHIBITION OF AMYLOID-β GENERATION IN MEMBRANE VESICLE-ADMINISTERED MICE

According to the following method, membrane vesicles of human adipose tissue-derived mesenchymal stem cells were administered to Alzheimer's disease model mice, and inhibition of amyloid-β generation was confirmed.

The exosome fraction prepared in the above-described manner was dissolved so that the total amount of the resultant mixture was 200 ml. This mixture was administered to the tail veins of mice (Alzheimer's disease model mice, one month old) immediately after the preparation. As a control, 200 ml of physiological saline was administered intravenously to littermate Alzheimer's disease model mice. The administration was performed 3 times in total at intervals of 7 days. Then, 7 days after the last administration, the animals were euthanized, and their brains were harvested and fixed. Then, tissue slices centered on the hippocampus, the cerebrum, and the thalamus were prepared, and amyloid-β plaques were stained with anti-amyloid-β antibody. Some of the collected and administered exosomes were subjected to protein extraction and the Western analysis, and the presence of a CD63 antigen as an exosome marker therein was confirmed.

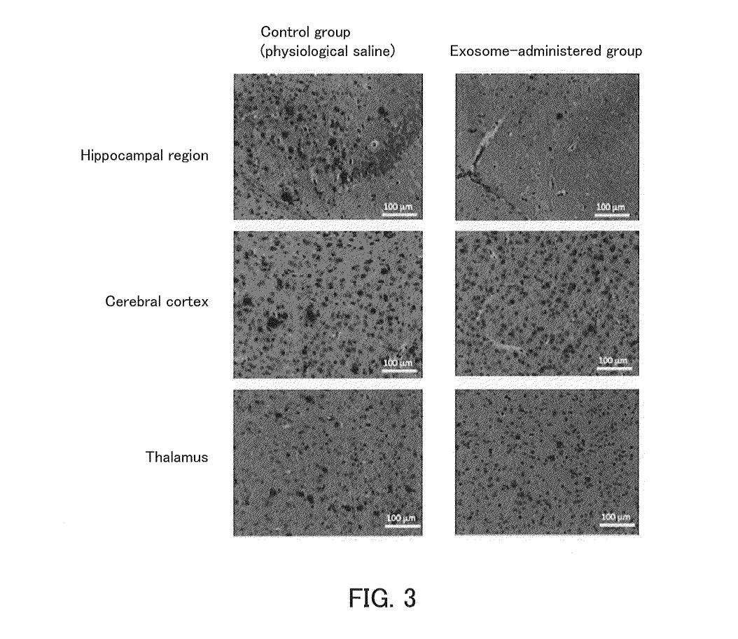

The photographs in FIG. 3 show inhibition of the amyloid-⊖ generation by the administration of the membrane vesicles. From FIG. 3, it can be seen that the generation of amyloid-β was inhibited in the brain of the model mouse to which the membrane vesicles had been administered.

1. A pharmaceutical product for preventing or treating Alzheimer's disease, the pharmaceutical product comprising:
a membrane vesicle of an adipose tissue-derived mesenchymal stem cell.

2. The pharmaceutical product according to claim 1, wherein the membrane vesicle comprises neprilysin.

3. A method for preventing or treating Alzheimer's disease, the method comprising:
administering a membrane vesicle of adipose tissue-derived mesenchymal stem cell to an Alzheimer's disease patient.

4. The method according claim 3, where
the membrane vesicle comprises neprilysin.
